SEQUENCE LISTING (8 sequences)

SEQ ID NO: 1 - DNA, 63 bp, Artificial Sequence
  Description of Artificial Sequence Primer
ggccagtgaattgtaatacgactcactatagggaggcggtttttttttttttttttttttttt

SEQ ID NO: 2 - DNA, 25 bp, Artificial Sequence
  Description of Artificial Sequence Primer
gtcgtcaagatgctaccgttcagga

SEQ ID NO: 3 - DNA, 802 bp, Homo sapiens
ggagggccgggcaccgcggcatggagggtcaacgctggctgccgctggaggccaatcccgaggtcaccaaccagtttcttaaacaattaggtctacatcctaactggcaattcgttgatgtatatggaatggatcctgaactccttagcatggtaccaagaccagtctgtgcagtcttacttctctttcctattacagaaaagtatgaagtattcagaacagaagaggaagaaaaaataaaatctcagggacaagatgttacatcatcagtatatttcatgaagcaaacaatcagcaatgcctgtggaacaattggactgattcatgctattgcaaacaataaagacaagatgcactttgaatctggatcaaccttgaaaaaattcctggaggaatctgtgtcaatgagccctgaagaacgagccagatacctggagaactatgatgccatccgagttactcatgagaccagtgcccatgaaggtcagactgaggcaccaagtatagatgagaaagtagatcttcattttattgcattagttcatgtagatgggcatctctatgaattagatgggcggaagccatttccaattaaccatggtgaaactagtgatgaaactttattagaggatgccatagaagtttgcaagaagtttatggagcgcgaccctgatgaactaagatttaatgcgattgctctttctgcagcatagcttgtcaataatggaaacaccaaaaactgtattatttgcaactaaattttctctgcccatacactaactcaaaaattttgatattttcc

SEQ ID NO: 4 - protein, 230 aa, Homo sapiens
MEGQRWLPLEANPEVTNQFLKQLGLHPNWQFVDVYGMDPELLSMVPRPVCAVLLLFPITEKYEVFRTEEEEKIKSQGQDVTSSVYFMKQTISNACGTIGLIHAIANNKDKMHFESGSTLKKFLEESVSMSPEERARYLENYDAIRVTHETSAHEGQTEAPSIDEKVDLHFIALVHVDGHLYELDGRKPFPINHGETSDETLLEDAIEVCKKFMERDPDELRFNAIALSAA

SEQ ID NO: 5 - DNA, 50 bp, Artificial Sequence
  Description of Artificial Sequence Primer
ggggacaagtttgtacaaaaaagcaggctatggagggtcaacgctggctg

SEQ ID NO: 6 - DNA, 50 bp, Artificial Sequence
  Description of Artificial Sequence Primer
ggggaccactttgtacaagaaagctgggtctatgctgcagaaagagcaat

SEQ ID NO: 7 - DNA, 866 bp, Homo sapiens
atggcggagcgcgggtacagcttttcgctgactacattcagcccgtctggtaaacttgtccagattgaatatgctttggctgctgtagctggaggagccccgtccgtgggaattaaagctgcaaatggtgtggtattagcaactgagaaaaaacagaaatccattctgtatgatgagcgaagtgtacacaaagtagaaccaattaccaagcatataggtttggtgtacagtggcatgggccccgattacagagtgcttgtgcacagagctcgaaaactagctcaacaatactatcttgtgtaccaagaacccattcctacagctcagctggtacagagagtagcttctgtgatgcaagaatatactcagtcaggtggtgttcgtccatttggagtttctttacttatttgtggttggaatgagggacgaccatatttatttcagtcagatccatctggagcttactttgcctggaaagctacagcaatgggaaagaactatgtgaatgggaagactttccttgagaaaagatataatgaagatctggaacttgaagatgccattcatacagccatcttaaccctaaaggaaagctttgaagggcaaatgacagaggataacatagaagttggaatctgcaatgaagctggatttaggaggcttactccaactgaagttaaggattacttggctgccatagcataacaatgaagtgactgaaaaatccagaatttcagataatctatctacttaaacatgtttaaagtatgttttgttttgcagactttttgcatacttatttctacatggtttaaatcgactgtttttaaaatgacacttataaatcctaataaactgttaaaccc

SEQ ID NO: 8 - protein, 234 aa, Homo sapiens
MAERGYSFSLTTFSPSGKLVQIEYALAAVAGGAPSVGIKAANGVVLATEKKQKSILYDERSVHKVEPITKHIGLVYSGMGPDYRVLVHRARKLAQQYYLVYQEPIPTAQLVQRVASVMQEYTQSGGVRPFGVSLLICGWNEGRPYLFQSDPSGAYFAWKATAMGKNYVNGKTFLEKRYNEDLELEDAIHTAILTLKESFEGQMTEDNIEVGICNEAGFRRLTPTEVKDYLAAIA

(19) United States
(12) Patent Application Publication
(10) Pub. No.: US 2002/0137099 A1
(43) Pub. Date: Sep. 26, 2002

(54) Methods for identifying substances for treating inflammatory conditions

(76) Inventors: Birgit Jung, Schwabenheim (DE); Stefan Mueller, Muenchen (DE); Norbert Kraut, Wien (AT)

Correspondence Address: BOEHRINGER INGELHEIM CORPORATION, 900 RIDGEBURY ROAD, P. O. BOX 368, RIDGEFIELD, CT, 06877, US

(21) Appl. No.: 10/029,630

(22) Filed: Dec. 21, 2001

Related U.S. Application Data
(60) Provisional application No. 60/257,856, filed on Dec. 22, 2000.

(51) Int. Cl. G01N 33/53

(52) U.S. Cl. 435/7.1

(57) ABSTRACT

The present invention relates to UDD-proteins involved in inflammatory processes and the modulation of the function of such a UDD-protein in order to positively influence inflammatory diseases.

### RELATED APPLICATION

The benefit of prior U.S. provisional application No. 60/257,856, filed Dec. 22, 2000 is hereby claimed.

### BACKGROUND

The present invention belongs to the field of modulation of inflammatory processes, in particular of chronic inflammatory airway diseases, in which macrophages play an important role. The inflammatory processes can be modulated according to the invention by influencing the biological activity of a protein mediating ubiquitin-dependent degradation, which protein is identified to be involved in the inflammatory process.

An example of chronic inflammatory airway disease, in which macrophages play an important role is chronic bronchitis (CB). CB may occur with or without airflow limitation and includes chronic obstructive pulmonary disease (COPD). CB is a complex disease encompassing symptoms of several disorders: chronic bronchitis which is characterized by cough and mucus hypersecretion, small airway disease, including inflammation and peribronchial fibrosis, emphysema, and airflow limitation. CB is characterized by an accelerated and irreversible decline of lung function. The major risk factor for developing CB is continuous cigarette smoking. Since only about 20% of all smokers are inflicted with CB, a genetic predisposition is also likely to contribute to the disease.

The initial events in the early onset of CB are inflammatory, affecting small and large airways. An irritation caused by cigarette smoking attracts macrophages and neutrophils the number of which is increased in the sputum of smokers. Perpetual smoking leads to an on-going inflammatory response in the lung by releasing mediators from macrophages, neutrophils and epithelial cells that recruit inflammatory cells to sites of the injury. So far there is no therapy available to reverse the course of CB. Smoking cessation may reduce the decline of lung function.

Only a few drugs are known to date to provide some relief for patients. Long-lasting β2-agonists and anticholinergics are applied to achieve a transient bronchodilation. A variety of antagonists for inflammatory events are under investigation, for example, LTB₄-inhibitors.

There is a continuous need to provide drugs for treating chronic inflammatory airway diseases. Chronic inflammatory airway diseases can be attributed to activated inflammatory immune cells, e.g. macrophages. There is, therefore, a need for drugs modulating the function of macrophages in order to eliminate a source of inflammatory processes.

### SUMMARY OF THE INVENTION

The present invention relates to methods for determining whether a substance is an activator or an inhibitor of a function of a UDD-protein comprising: (a) contacting the UDD-protein, or functionally equivalent mutants, variants, and fragments thereof, with a substance to be tested; and (b) measuring whether the function is inhibited or activated. The invention encompasses measuring such functions directly or indirectly, and using a cellular or cell-free system. The methods further encompass using mammalian or human protein. In an embodiment, the UDD-protein consists of an amino acid sequence selected form the group consisting of: SEQ ID NO:4 and SEQ ID NO:8. Preferably, the UDD-protein is SEQ ID NO:4 or a functionally equivalent variant, mutant or fragment thereof or SEQ ID NO:8 or a functionally equivalent variant, mutant or fragment thereof. The functions measured by the methods of the invention include substrate binding.

The present invention also relates to methods for determining an expression level of a UDD-protein comprising: (a) determining the level of the UDD-protein expressed in a hyperactivated macrophage; (b) determining the level of the UDD-protein expressed in a non-hyperactivated macrophage; and (c) comparing the level of the UDD-protein expressed in step (a) to the level of the UDD-protein expressed in step (b), wherein a difference in levels indicates a differentially expressed UDD-protein, as well as functionally equivalent mutants, variants, and fragments of a UDD-protein, in particular, an amino acid sequence of SEQ ID NOs:4 and/or 8, as well as functionally equivalent mutants, variants, and fragments thereof. The level may be determined on a protein or nucleic acid level.

The present invention also relates to methods for diagnosing or monitoring a chronic inflammatory airway disease comprising: (a) determining the level of a UDD-protein expressed in a hyperactivated macrophage; (b) determining the level of the UDD-protein expressed in a non-hyperactivated macrophage; and (c) comparing the level of the UDD-protein expressed in step (a) to the level of the UDD-protein expressed in step (b), wherein a difference in levels indicates a differentially expressed UDD-protein. The level may be determined on a protein or nucleic acid level. In an embodiment, the macrophage or a part thereof used in such methods is obtainable from the site of inflammation. The method further encompasses diagnosing or monitoring a chronic inflammatory airway disease wherein the disease is selected from the group consisting of: CB and COPD.

The present invention also relates to methods for treating a chronic inflammatory airway disease comprising: administering to a subject in need of such treatment an effective amount of a pharmaceutical composition comprising at least one substance determined to be an activator or an inhibitor of a UDD-protein. Such substances may be determined to be activators or inhibitors using the methods of the invention. Preferably, the subject is a mammal, more preferably a human. Preferably, the chronic inflammatory airway disease is selected from the group consisting of: CB and COPD.

The present invention also relates to methods for selectively modulating a UDD-protein in a macrophage, comprising administering a substance determined to be an activator or an inhibitor of a UDD-protein. Such substances may be determined to be activators or inhibitors using the methods of the invention. The methods further encompass wherein the macrophage is involved in a chronic inflammatory airway disease preferably selected from the group consisting of: CB and COPD.

The present invention also relates to substances determined to be activators or inhibitors of a UDD-protein. Such substances may be determined to be activators or inhibitors using the methods of the invention. Such substances of the invention may be useful for treating a chronic inflammatory airway disease, preferably selected from the group consisting of: CB and COPD.

The invention also encompasses pharmaceutical compositions of such substances.

### DESCRIPTION OF THE INVENTION

In the present invention it was found that macrophages involved in an inflammatory process, particularly in a chronic inflammatory airway disease, more particularly in chronic bronchitis or COPD, show a pattern of differentially expressed nucleic acid sequence and protein expression which differs from the pattern of gene expression of macrophages from healthy donors or donors in an irritated state, which latter do contain macrophages in an activated state. Therefore, macrophages show different activation levels under different inflammatory conditions. For example, it is shown in the present invention that macrophages involved in an inflammatory process in COPD smokers show different gene expression pattern than macrophages from healthy smokers, indicating that in COPD smokers macrophages are in a different, hereinafter named “hyperactivated” or “hyperactive” state. The present invention provides for the inhibition of the hyperactivation or the reduction of the hyperactive state of a macrophage by the identification of substances which modulate a protein mediating ubiquitin-dependent degradation involved in the hyperactivation or maintenance of the hyperactive state.

The term “chronic inflammatory airway disease” as used hereinafter includes but is not limited to, Chronic Bronchitis (CB) and Chronic Obstructive Pulmonary Disease (COPD). The preferred meaning of the term “chronic inflammatory airway disease” is CB and COPD; the more preferred meaning is CB or COPD.

The term “a” as used herein refers to one or more, e.g., “a” UDD-protein refers to one or more UDD-proteins.

The invention is based on the identification of a nucleic acid sequence differentially expressed in a hyperactivated macrophage compared to a macrophage which is not hyperactivated. Such a nucleic acid sequence encodes a protein which mediates ubiquitin-dependent degradation, which protein is involved in the hyperactivation or maintenance of the hyperactive state of a macrophage involved in an inflammatory process, preferably in a chronic inflammatory airway disease. Such differentially expressed nucleic acid sequence or protein encoded by such nucleic acid sequence is also referred to hereinafter as differentially expressed nucleic acid sequence or protein of the invention, respectively. In particular, the present invention teaches a link between phenotypic changes in macrophages due to differentially expressed nucleic acid sequence and protein expression pattern and involvement of macrophages in inflammatory processes and, thus, provides a basis for a variety of applications. For example, the present invention provides a method and a test system for determining the expression level of a macrophage protein of the invention or differentially expressed nucleic acid sequence of the invention and thereby provides, e.g. for methods for diagnosis or monitoring of inflammatory processes with involvement of hyperactivated macrophages in mammalian, preferably human beings, especially such beings suffering from an inflammatory process, preferably in a chronic inflammatory airway disease, more preferably in chronic bronchitis or COPD. The invention also relates to a method for identifying a substance by means of a differentially expressed nucleic acid sequence or protein of the invention, which substance modulates, i.e. acts as an inhibitor or activator on the said differentially expressed nucleic acid sequence or protein of the invention and thereby positively influences chronic inflammatory processes by inhibition of the hyperactivation or reduction of the hyperactive state of macrophages, and thereby allows treatment of mammals, preferably human beings, suffering from a said disease. The invention also relates to a method for selectively modulating such a differentially expressed nucleic acid sequence or protein of the invention in a macrophage comprising administering a substance determined to be a modulator of said protein or differentially expressed nucleic acid sequence. The present invention includes the use of said substances for treating beings in need of a treatment for an inflammatory process.

In the present invention in a first step, a differentially expressed nucleic acid sequence of the invention is identified which has a different expression pattern in a hyperactivated macrophage compared to a macrophage which is not hyperactivated. For the sake of conciseness, this description deals particularly with investigation of macrophages involved in COPD; however, equivalent results may be obtained with samples from subjects suffering from other chronic inflammatory airway diseases, e.g. other chronic bronchitis symptoms. The investigation of the different expression pattern leads to the identification of a series of differentially expressed nucleic acid sequences expressed in dependency on the activation state of a macrophage involved in an inflammatory process, as exemplified in the Examples hereinbelow.

Briefly, such a differentially expressed nucleic acid sequence of the invention is identified by comparative expression profiling experiments using a cell or cellular extract from a hyperactivated macrophage, i.e. for example from the site of inflammation in COPD and from the corresponding site of control being not suffering from said disease, however, suffering under the same irritating condition such as cigarette smoke exposure.

In a second step the proteins are identified which are encoded by the differentially expressed nucleic acid sequence, i.e. proteins playing a role in mediating the hyperactivation or in maintaining the hyperactivated state. A class of differentially expressed nucleic acid sequences of the invention can be identified to encode a class of proteins which mediate ubiquitin-dependent degradation which is characterized in that it is expressed in a macrophage that is hyperactivated according to the invention at a lower or higher level than the control level in a macrophage which is not hyperactivated. Such a protein of the invention is hereinafter named UDD-protein (“protein mediating ubiquitin-dependent degradation” and which is deregulated in a hyperactive macrophage).

A preferred example of a UDD-protein according to the present invention is UCH-L3 (Larsen, C. N. et al. (1998) Biochemistry 37, 3358-3368), or proteasome subunit HC3 (Tiao, G. et al. (1997) J. Clin. Invest. 99,163-168; Hobler, S. C. et al. (1999) Am. J. Physiol. 277, R434-R440), depicted in the sequence listing.

The biological activity of a UDD-protein according to the present invention, i.e. mediating the involvement of a macrophage in an inflammatory process according to the invention, is dependent, for example, on recognition of proteins conjugated with ubiquitin and/or on other UDD-protein functions such as protease activity or any other function of the respective UDD-protein relevant for its biological activity.

The invention also concerns functional equivalents, derivatives, variants, mutants and fragments of a UDD-protein, preferentially of the preferred proteins mentioned hereinbefore. Functional in this context means having a function of the respective corresponding UDD-protein which is involved in its biological activity, e.g. substrate recognition.

According to the present invention, the biological activity of a UDD-protein expressed at a lower level than the control level is preferably activated in order to inhibit hyperactivation or reduce a hyperactivated state of a macrophage, whereby the biological activity of a UDD-protein which is expressed at a higher level than the control level is preferably inhibited in order to inhibit hyperactivation or reduce a hyperactivated state of a macrophage.

In one embodiment, the present invention concerns a test method for determining a substance to be an activator or inhibitor of a UDD-protein. In one embodiment, the present invention concerns a test method for determining whether a substance is an activator or inhibitor of a UDD-protein. Since a UDD-protein is involved in a chronic inflammatory airway disease and plays a role in mediating inflammation, a substance modulating the biological activity of a UDD-protein can be used for treating a chronic inflammatory airway disease or can be used as a lead compound for optimization of the function of the substance in a way that the optimized substance is suitable for treating chronic inflammatory airway diseases. For performing a method of the invention, a test system according to the invention can be used.

The present invention also concerns a test system for determining whether a substance is an activator or an inhibitor of a UDD-protein function. A test system useful for performing a method of the invention comprises a cellular or a cell-free system. For example, one embodiment of the invention concerns a test system that is designed in a way to allow the testing of substances acting on the expression level of the differentially expressed nucleic acid sequence e.g. using expression of a reporter-gene, e.g. luciferase gene or the like, as a measurable readout. Another embodiment of the invention concerns a test system that is designed in a way to allow the testing of substances directly interacting with a function, e.g. the enzymatic activity, of the UDD-protein or interfering with the activation of a function, e.g. enzymatic activity, of the UDD-protein by a natural or an artificial but appropriate activator of the UDD-protein, e.g. an appropriate kinase or the like.

A test system of the invention comprises, for example, elements well known in the art. For example, cell-free systems may include a UDD-protein or a functional equivalent, derivative, variant, mutant or fragment of a UDD-protein, a nucleic acid encoding a UDD-protein or encoding a functional equivalent, derivative, variant, mutant or fragment of a UDD-protein in soluble or bound form or in cellular compartments or vesicles. Suitable cellular systems include, for example, a suitable prokaryotic cell or eukaryotic cell, e.g. such comprising a UDD-protein or a functional equivalent, derivative, variant, mutant or fragment of a UDD-protein, a nucleic acid encoding a UDD-protein or encoding a functional equivalent, derivative, variant, mutant or fragment of UDD-protein (Tsuchiya, S. et al. (1980) Int. J. Cancer 26, 171-176; Ziegler-Heitbrock, H. W. et al. (1988) Int. J. Cancer 41, 456-461). A cell suitable for use in a said test system of the invention may be obtained by recombinant techniques, e.g. after transformation or transfection with a recombinant vector suitable for expression of a desired UDD-protein or functional equivalent, derivative, variant, mutant or fragment of a UDD-protein, or may be, e.g. a cell line or a cell isolated from a natural source expressing a desired UDD-protein or functional equivalent, derivative, variant, mutant or fragment of UDD-protein. A test system of the invention may include a natural or artificial ligand of a UDD-protein if desirable or necessary for testing whether a substance of interest is an inhibitor or activator of a UDD-protein.

A test system of the invention comprises, for example, elements well known in the art. Cell-free systems may include, for example, cellular compartments or vesicles comprising a UDD-protein. Suitable cellular systems include, for example, a suitable prokaryotic cell or eukaryotic cell, e.g. such comprising a UDD-protein. A cell suitable for use in a said test system of the invention may be obtained by recombinant techniques, e.g. after transformation or transfection with a vector suitable for expression of the desired UDD-protein, or may be, e.g. a cell line or a cell isolated from a natural source expressing the desired UDD-protein. A test system of the invention may include a natural or artificial ligand of the UDD-protein if desirable or necessary for testing whether a substance of interest is an inhibitor or activator of a UDD-protein.

A test method according to the invention comprises measuring a read-out, e.g. a phenotypic change in the test system, for example, if a cellular system is used, a phenotypic change of the cell is monitored. Such change may be a change in a naturally occurring or artificial response, e.g. a reporter gene expression of the cell to UDD-protein activation or inhibition, e.g. as detailed in the Examples hereinbelow.

A test method according to the invention can on the one hand be useful for high throughput testing suitable for determining whether a substance is an inhibitor or activator of the invention, but also e.g. for secondary testing or validation of a hit or lead substance identified in high throughput testing.

The present invention also concerns a substance identified in a method according to the invention to be an inhibitor or activator of a UDD-protein. A substance of the present invention is any compound which is capable of activating or preferably inhibiting a function of a UDD-protein according to the invention. An example of a way to activate or inhibit a function of a UDD-protein is by influencing the expression level of said UDD-protein. Another example of a way to activate or inhibit a function of a UDD-protein is to apply a substance which directly binds the UDD-protein and thereby activates or blocks functional domains of said UDD-protein, which can be done reversibly or irreversibly, depending on the nature of the substance applied.

Accordingly, a substance useful for activating or inhibiting biological activity of a UDD-protein includes a substance acting on the expression of differentially expressed nucleic acid sequence, for example a nucleic acid fragment hybridizing with the corresponding gene or regulatory sequence and thereby influencing gene expression, or a substance acting on a UDD-protein itself or on its activation or inhibition by other naturally occurring cellular components, e.g. an other protein acting enzymatically on a UDD-protein, e.g. a protein kinase.

Therefore, the invention concerns, for example, a substance which is a nucleic acid sequence coding for the gene of a UDD-protein, or a fragment, derivative, mutant or variant of such a nucleic acid sequence, which nucleic acid sequence or a fragment, derivative, mutant or variant thereof is capable of influencing the gene expression level, e.g. a nucleic acid molecule suitable as antisense nucleic acid, ribozyme, or for triple helix formation.

The invention also concerns a substance which is e.g. an antibody or an organic or inorganic compound which directly binds to or interferes with the activation of a UDD-protein and thereby affects its biological activity.

In a further aspect, the present invention relates to a method for determining an expression level of a nucleic acid coding for a UDD-protein, preferably messenger RNA, or a UDD-protein itself, in a cell, preferably in a macrophage, more preferably in a macrophage isolated from a site of inflammation, even more preferably from a site of inflammation in a subject suffering from a chronic inflammatory airway disease. Such a method can be used, for example, for testing whether a substance is capable of influencing differentially expressed nucleic acid sequence expression levels in a method outlined above for determining whether a substance is an activator or inhibitor according to the present invention. A method for determining an expression level according to the invention can, however, also be used for testing the activation state of a macrophage, e.g. for diagnostic purposes or for investigation of the success of treatment for a disease which is caused by the hyperactivated macrophage. Said macrophage is preferably a mammalian, more preferably a human cell. Accordingly, macrophages of the present invention are preferably obtainable from the site of inflammation in a mammal and more preferably from a site of inflammation in a human being. Accordingly, the invention also relates to a method for diagnosis of a chronic inflammatory disease, or monitoring of such disease, e.g. monitoring success in treating beings in need of treatment for such disease, comprising determining an expression level of a nucleic acid coding for a UDD-protein, preferably messenger RNA, or a UDD-protein itself in a macrophage.

The present invention also relates to the use of a substance according to the invention for the treatment of a chronic inflammatory airway disease. Another embodiment of the present invention relates to a pharmaceutical composition comprising at least one of the substances according to the invention determined to be an activator or an inhibitor. The composition may be manufactured in a manner that is itself known, e.g. by means of conventional mixing, dissolving, granulating, dragee-making, levigating, powdering, emulsifying, encapsulating, entrapping or lyophilizing processes.

In order to use substances which activate or inhibit according to the invention as drugs for treatment for chronic inflammatory airway diseases, the substances can be tested in animal models, for example, an animal suffering from an inflammatory airway disorder or a transgenic animal expressing a UDD-protein according to the invention.

Toxicity and therapeutic efficacy of a substance according to the invention can be determined by standard pharmaceutical procedures, which include conducting cell culture and animal experiments to determine the IC₅₀, LD₅₀ and ED₅₀. The data obtained are used for estimating the animal or more preferred the human dose range, which will also depend on the dosage form (tablets, capsules, aerosol sprays ampules, etc.) and the administration route (for example transdermal, oral, buccal, nasal, enteral, parenteral, inhalative, intratracheal, or rectal).

A pharmaceutical composition containing at least one substance according to the invention as an active ingredient can be formulated in conventional manner. Methods for making such formulations can be found in manuals, e.g. “Remington Pharmaceutical Science”. Examples for ingredients that are useful for formulating at least one substance according to the present invention are also found in WO 99/18193, which is hereby incorporated by reference.

In a further aspect the invention concerns a method for treating a chronic inflammatory airway disease according to the invention. Such method comprises administering to a being, preferably to a human being, in need of such treatment a suitable amount of a pharmaceutical composition comprising at least one substance determined to be an activator or an inhibitor by a method according to the invention for determining whether a substance is an activator or an inhibitor of a UDD-protein according to the invention.

In an other embodiment the invention relates to a method for selectively modulating UDD-protein concentration in a macrophage, comprising administering a substance determined to be an activator or inhibitor of a UDD-protein according to the invention.

Included herein are exemplified embodiments, which are intended as illustrations of single aspects of the invention. Indeed, various modifications of the invention in addition to those herein will become apparent to those skilled in the art from the foregoing description. Such modifications are intended to fall within the scope of the present invention.

All publications and patent applications cited herein are incorporated by reference in their entireties.

### EXAMPLES

### Example 1

### Comparative Expression Profiling

The following is an illustration of how comparative expression profiling can be performed in order to identify a UDD-protein

1.1. Selection of Patients

Three groups of subjects are studied: healthy non-smokers, healthy smokers and patients with COPD.

In order to assess lung function, subjects have to perform spirometry. A simple calculation based on age and height is used to characterize the results. COPD subjects are included if their FEV₁% (forced expiratory volume, 1 second) predicted is less than 70%. Healthy smokers are age and smoking history matched with the COPD subjects but have normal lung function. Healthy non-smokers have normal lung function and have never smoked. The latter group has a methacholine challenge to exclude asthma. This technique requires increasing doses of methacholine to be given to the subject, with spirometry between each dose. When the FEV₁ falls 20% the test is stopped and the PC₂₀ is calculated. This is the dose of methacholine causing a 20% fall in FEV₁ and we require a value of greater than 32 as evidence of absence of asthma. All subjects have skin prick tests to common allergens and are required to have negative results. This excludes atopic individuals. The clinical history of the subjects is monitored and examined in order to exclude concomitant disease.

1.2. BAL (Bronchoalveolar Ravage) Procedure

Subjects are sedated with midazolam prior to the BAL. Local anaesthetic spray is used to anesthetize the back of the throat. A 7 mm Olympus bronchoscope is used. The lavaged area is the right middle lobe. 250 ml of sterile saline is instilled and immediately aspirated. The resulting aspirate contains macrophages.

1.3. BAL Processing

BAL is filtered through sterile gauze to remove debris. The cells are washed twice in HBSS, resuspended in 1 ml HBSS (Hank's Balanced Salt Solution) and counted. The macrophages are spun to a pellet using 15 ml Falcon blue-cap polypropylene, resuspended in Trizol reagent (Gibco BRL Life Technologies) at a concentration of 1 ml Trizol reagent per 10 million cells and then frozen at −70° C.

1.4. Differential Gene Expression Analysis

Total RNA is extracted from macrophage samples obtained according to Example 1.3. Cell suspensions in Trizol are homogenized through pipetting and incubated at room temperature for 5 minutes. 200 μl chloroform per ml Trizol is added, the mixture carefully mixed for 15 seconds and incubated for 3 more minutes at room temperature. The samples are spun at 10,000 g for 15 minutes at 4° C. The upper phase is transferred into a new reaction tube, and the RNA is precipitated by adding 0.5 ml isopropanol per ml Trizol for 10 minutes at room temperature (RT). Then, the precipitate is pelleted by using a microcentrifuge for 10 minutes at 4° C. with 10,000 g, the pellet is washed twice with 75% ethanol, air dried and resuspended in DEPC-H₂O.

An RNA cleanup with Qiagen RNeasy Total RNA isolation kit (Qiagen) is performed in order to improve the purity of the RNA. The purity of the RNA is determined by agarose gel electrophoresis and the concentration is measured by UV absorption at 260 nm.

5 μg of each RNA is used for cDNA synthesis. First and second strand syntheses are performed with the SuperScript Choice system (Gibco BRL Life Technologies). In a total volume of 11 μl RNA and 1 μl of 100 μM T7-(dt)₂₄ primer, sequence shown in SEQ ID NO. 1, are heated up to 70° C. for 10 minutes and then cooled down on ice for 2 minutes. First strand buffer to a final concentration of 1×. DTT to a concentration of 10 mM and a dNTP mix to a final concentration of 0.5 mM are added to a total volume of 18 μl. The reaction mix is incubated at 42° C. for 2 minutes and 2 μl of Superscript II reverse transcriptase (200 U/μl) are added. For second strand synthesis, 130 μl of a mix containing 1.15×second strand buffer, 230 μM dNTPs, 10 U E. coli DNA ligase (10U/μl), E. coli DNA polymerase (10 U/μl), RNase H (2U/μl) is added to the reaction of the first strand synthesis and carefully mixed with a pipette. Second strand synthesis is performed at 16° C. for 2 hours, then 2 μl of T4 DNA polymerase (5 U/μl) are added, incubated for 5 minutes at 16° C. and the reaction is stopped by adding 10 μl 0.5 M EDTA.

Prior to cRNA synthesis, the double stranded cDNA is purified. The cDNA is mixed with an equal volume of phenol:chloroform:isoamylalcohol (25:24:1) and spun through the gel matrix of phase lock gels (Eppendorf) in a microcentrifuge in order to separate the cDNA from unbound nucleotides. The aqueous phase is precipitated with ammonium acetate and ethanol. Subsequently, the cDNA is used for in vitro transcription. cRNA synthesis is performed with the ENZO BioArray High Yield RNA Transcript Labeling Kit according to manufacturer's protocol (ENZO Diagnostics). Briefly, the cDNA is incubated with 1×HY reaction buffer, 1×biotin labeled ribonucleotides, 1×DTT, 1×RNase Inhibitor Mix and 1×T7 RNA Polymerase in a total volume of 40 μl for 5 hours at 37° C. Then, the reaction mix is purified via RNeasy columns (Qiagen), the cRNA precipitated with ammonium acetate and ethanol and finally resuspended in DEPC-treated water. The concentration is determined via UV spectrometry at 260 nm. The remaining cRNA is incubated with 1×fragmentation buffer (5×fragmentation buffer: 200 mM Tris acetate, pH 8.1, 500 mM KOAc, 150 mM MgOAc) at 94° C. for 35 minutes.

For hybridization of the DNA chip, 15 μg of cRNA is used, mixed with 50 pM biotin-labeled control B2 oligonucleotide, sequence shown SEQ ID NO:2, 1×cRNA cocktail, 0.1 mg/ml herring sperm DNA, 0.5 mg/ml acetylated BSA, 1×MES (2-[N-morpholino]-ethanesulfonic acid) hybridization buffer in a total volume of 300 μl. The hybridization mixture is heated up to 99° C. for 5 minutes, cooled down to 45° C. for 10 minutes and 200 μl of the mix are used to fill the probe array. The hybridization is performed at 45° C. at 60 rpm for 16 hours.

After the hybridization, the hybridization mix on the chip is replaced by 300 μl non-stringent wash buffer (100 mM MES, 100 mM NaCl, 0.01% Tween 20). The chip is inserted into an Affymetrix Fluidics station and washing and staining is performed according to the EukGE-WS2 protocol. The staining solution per chip consists of 600 μl 1×stain buffer (100 mM MES, 1 M NaCl, 0.05% Tween 20), 2 mg/ml BSA, 10 μg/ml SAPE (streptavidin phycoerythrin) (Dianova); the antibody solution consists of 1×stain buffer, 2 mg/ml BSA, 0.1 mg/ml goat IgG, 3 μg/ml biotinylated antibody.

After the washing and staining procedure the chips are scanned on the HP Gene Array Scanner (Hewlett Packard).

Data Analysis is performed by pair-wise comparisons between chips hybridized with RNA isolated from COPD smokers and chips hybridized with RNA isolated from healthy smokers.

The following is an illustration of differentially expressed genes and their function as identified according to the approach of the present invention.

### Example 2

### UCH-L3

A gene that is identified as being up-regulated in COPD smokers compared to healthy smokers is the ubiquitin carboxyl-terminal hydrolase isozyme L3 (UCH-L3)(SEQ ID NOs:3,4).

UCH-L3 is a thiol protease that binds tightly to ubiquitin. Preferring small molecular weight ubiquitin adducts (such as amino acids or oligopeptides), it recognizes and hydrolyzes a peptide bond at the C-terminus of ubiquitin with high efficiency and low sequence preference. UCH-L3 may function to regenerate ubiquitin from attached polypeptides (Larsen, C. N. et al. (1998) Biochemistry 37, 3358-3368).

UCH-L3 is consistently found up-regulated (52%) in comparisons between COPD smokers and healthy smokers. This is shown by “fold change” values (Table 1). The p values for two separate groups comparing COPD smokers and healthy smokers are 0.01 and 0.29.

TABLE 1
Fold change values (FC) for comparisons between obstructed
smoker and healthy smokers. On average UCH-L3 is up-regulated
by 1.8 fold, the median is 2 fold

| comp | FC | comp | FC | comp | FC | comp | FC |
|---|---|---|---|---|---|---|---|
| 1 vs 2 | 1.1 | 5 vs 43 | 1.0 | 39 vs 57 | 2.9 | 68 vs 66 | 1.8 |
| 1 vs 37 | 1.4 | 5 vs 56 | 1.0 | 39 vs 58 | 2.9 | 68 vs 69 | 3.6 |
| 1 vs 43 | 2.3 | 5 vs 57 | 1.0 | 39 vs 62 | 2.6 | 68 vs 76 | 2.1 |
| 1 vs 56 | 2.4 | 5 vs 58 | 1.0 | 44 vs 2 | −1.1 | 68 vs 78 | 1.8 |
| 1 vs 57 | 2.6 | 5 vs 62 | 1.0 | 44 vs 37 | 1.4 | 70 vs 65 | −1.2 |
| 1 vs 58 | 2.4 | 6 vs 2 | 1.6 | 44 vs 43 | 1.8 | 70 vs 66 | 1.1 |
| 1 vs 62 | 2.0 | 6 vs 37 | 2.3 | 44 vs 56 | 2.1 | 70 vs 69 | 2.5 |
| 3 vs 2 | 1.4 | 6 vs 43 | 2.6 | 44 vs 57 | 2.1 | 70 vs 76 | 1.4 |
| 3 vs 37 | 2.2 | 6 vs 56 | 2.9 | 44 vs 58 | 2.1 | 70 vs 78 | 1.2 |
| 3 vs 43 | 3.0 | 6 vs 57 | 2.8 | 44 vs 62 | 1.9 | 71 vs 65 | 1.1 |
| 3 vs 56 | 3.3 | 6 vs 58 | 2.8 | 64 vs 65 | −1.5 | 71 vs 66 | 1.7 |
| 3 vs 57 | 3.3 | 6 vs 62 | 2.7 | 64 vs 66 | −1.2 | 71 vs 69 | 3.4 |
| 3 vs 58 | 3.3 | 39 vs 2 | 1.4 | 64 vs 69 | 2.0 | 71 vs 76 | 2.0 |
| 3 vs 62 | 2.6 | 39 vs 37 | 2.1 | 64 vs 76 | 1.1 | 71 vs 78 | 1.7 |
| 5 vs 2 | −1.2 | 39 vs 43 | 2.7 | 64 vs 78 | 1.0 | | |
| 5 vs 37 | 1.1 | 39 vs 56 | 3.0 | 68 vs 65 | 1.2 | | |

2.1. Cloning of UCH-L3

UCH-L3 is cloned from a total RNA extracted from human PMNs (polymorphonuclear neutrophils) isolated from healthy volunteers. 5 μg RNA is reverse transcribed into cDNA with 5 ng oligo(dt)₁₈ primer, 1×first strand buffer, 10 mM DTT, 0.5 mM dNTPs and 2 U Superscript II (Gibco BRL) at 42° C. for 50 minutes. Then, the reaction is terminated at 70° C. for 15 minutes and the cDNA concentration is determined by UV-spectrophotometry. For amplification of UCH-L3, 100 ng of the cDNA and 10 pmoles of sequence-specific primers for UCH-L3 (forward primer, SEQ ID NO:5 and reverse primer, SEQ ID NO:6) are used for PCR (polymerase chain reaction). Reaction conditions are: 2 minutes at 94° C., 35 cycles with 30 seconds at 94° C., 30 seconds at 53° C., 90 seconds at 72° C., followed by 7 minutes at 72° C. with Taq DNA-polymerase. The reaction mix is separated on a 2% agarose gel, a band of about 1,000 bp is cut out and purified with the QIAEX II extraction kit (Qiagen). The concentration of the purified band is determined and about 120 ng are incubated with 300 ng of pDONR201, the donor vector of the Gateway system (Life Technologies), 1×BP clonase reaction buffer, BP clonase enzyme mix in a total volume of 20 μl for 60 minutes at 25° C. Then, reactions are incubated with 2 μl of proteinase K and incubated for 10 minutes at 37° C. The reaction mix is then electroporated into competent DB3.1 cells and plated on Kanamycin-containing plates. Clones are verified by sequencing. A clone, designated pDONR-UCHL3, with identical sequence to the database entry (acc. M30496) is used for further experiments.

2.2. UCH-L3 Expression Vector

The vector containing UCH-L3 described under 1.1. is used to transfer the cDNA for UCH-L3 to the expression vector pcDNA3.1 (+)/attR that contains the “attR1” and “attR2” recombination sites of the Gateway cloning system (Life Technologies) where UCH-L3 is expressed under the control of the CMV promoter. 150 ng of the “entry vector” pDONR-UCH-L3 is mixed with 150 ng of the “destination vector” pcDNA3.1 (+)/attR, 4 μl of the LR Clonase enzyme mix, 4 μl LR Clonase reaction buffer, added up with TE (Tris/EDTA) to 20 μl and incubated at 25° C. for 60 minutes. Then, 2 μl of proteinase K solution is added and incubated for 10 minutes at 37° C. 1 μl of the reaction mix is transformed into 50 μl DH5α by a heat-shock of 30 seconds at 42° C. after incubating cells with DNA for 30 minutes on ice. After heat-shock of the cells 450 μl of S.O.C. is added and cells are incubated at 37° C. for 60 minutes. Cells (100 μl) are plated on LB plates containing 100 μg/ml ampicillin and incubated overnight.

A colony that contains pcDNA3.1 (+)/attR with UCH-L3 as an insert is designated pcDNA/UCHL3 and used for transfection studies.

2.3. Expression of Recombinant UCH-L3

The vector containing UCH-L3 described under 1.1. is used to transfer the cDNA for UCH-L3 to the expression vectors gpET28abc/attR that contains the “attR1” and “attR2” recombination sites of the Gateway cloning system (Life Technologies). These vectors allow the expression of recombinant his-tagged UCH-L3 in bacteria under the control tog the T7 promoter. 150 ng of the “entry vector” pDONR-UCH-L3 is mixed with 150 ng of the “destination vector” gpET28abc/attR, 4 μl of the LR Clonase enzyme mix, 4 μl LR Clonase reaction buffer, added up with TE (Tris/EDTA) to 20 μl, and incubated at 25° C. for 60 minutes. Then, 2 μl of proteinase K solution is added and incubated for 10 minutes at 37° C. 1 μl of the reaction mix is transformed into 50 μl DH5α by a heat-shock of 30 seconds at 42° C. after incubating cells with DNA for 30 minutes on ice. After heat-shock of the cells, 450 μl, of S.O.C. is added and cells are incubated at 37° C. for 60 minutes. Cells (100 μl) are plated on LB plates containing 100 μg/ml ampicillin and incubated overnight.

A colony that contains gpET28abc/attR with UCH-L3 fused to the his-tag in the correct reading frame is designated pgPET/UCHL3 and used for expression of UCH-L3 in bacteria.

2.4. Purification of Recombinant UCH-L3

One liter LB broth including 100 μg/ml ampicillin is inoculated with 0.5 ml of an overnight culture of E. coli M15(pREP4) that carries pQE/ARL4. The culture is incubated at 37° C. with vigorous shaking until OD₆₀₀ of 0.6. Expression is induced by adding 1 mM IPTG and the culture is grown further for 4 hours. Cells are harvested by centrifugation at 4,000×g for 20 minutes at 4° C. The pellet is frozen at −20° C.

Cells are thawed on ice and resuspended in 2 ml/g cell pellet of lysis buffer (50 mM NaH₂PO4, pH 8.0, 300 mM NaCl, 10 mM imidazole). Then, lysozyme is added to 1 mg/ml and incubated on ice for 30 minutes. Then, cells are sonicated (six bursts of 10 seconds at 300 W). 10 μg/ml RNase A and 5 μg/ml DNase I is added and incubated on ice for 10 minutes. Then, lysates are cleared by spinning debris at 1,000×g for 20 minutes at 4° C. Then, protease inhibitors (40 μg/ml bacitracin, 4 μg/ml leupeptin, 4 μg/ml chymostatin, 10 μg/ml pefabloc, 100 μM PMSF) are added. 3 ml of Ni-NTA resin (Qiagen) are added to the lysate and filled into a column. Binding to the resin is allowed for 60 minutes at 4° C. during gentle shaking. Then, column outlet is opened, the resin washed twice with 12 ml wash buffer (50 mM NaH₂PO4, pH 8.0, 300 mM NaCl, 20 mM imidazole) and eluted with four times 3 ml of elution buffer (50 mM NaH₂PO4, pH 8.0, 300 mM NaCl, 250 mM imidazole). The elution fraction that contains the recombinant protein is determined by SDS-PAGE and protein concentration of the purified protein is determined by the method of Bradford.

2.5. SPA (Scintillation Proximity Assay) as Activity Assay

### Materials

The assay is performed in 384-well plates (Packard Optiplate, white, flat bottom, Prod.-No. 6005214). Ubiquitinyl-L-Asp with a biotin at the N-terminus of ubiquitin and tritiated aspartate is used as a substrate. Recombinant UCH-L3 is stored in 50 mM Tris/HCl, pH7.6, 5 mM DTT, 50 μg/ml ovalbumin at −80° C.

### Method

In the 384-well plates 10 μl test compound in demineralized water (containing 5% DMSO, final concentration 1%) are mixed with 20 μl of 10 nM biotin-Ubiquitine-L-³H-Asp in 50 mM Tris/Cl pH7.6, 5 mM DTT, 50 mM ovalbumin. For the “negative” controls (100% CTL, completely inhibited enzyme activity), the test compound is replaced by 2-phosphono-methyl-pentanedioic acid (PMPA, 500 nM, f.c. 100 nM). For the “positive” controls (0% CTL, non-inhibited enzyme activity), the test compound is omitted from the above mixture. The UCH-L3 preparation is 20×diluted in demineralized water and 20 μl of this diluted enzyme solution are added to each well. The plates are then incubated at 37° C. for 1 hour. After the incubation period, 0.05 mg/well of LEADseeker streptavidin-coated polystyrene beads are added in 30 μl of 0.375 M KH₂PO₄. After 2 h of incubation at RT, the plates are measured in the LEADseeker.

Each assay microtiter plate contains wells with “negative” and “positive” controls as described above. The analysis of the data is performed by the calculation of the percentage of scintillation in the presence of the test compound compared to the scintillation of the “negative” control after subtracting the “positive” control:

% CTL=(scintillation (“negative” control)−scintillation (sample))*100/(scintillation (“negative” control)−scintillation (“positive” control)).

An inhibitor of UCH-L3 will give values between 100% CTL (no inhibition) and 0% CTL (complete inhibition). Values of less than 0% CTL are normally related to compound-specific physico-chemical properties or indirect biochemical effects such as allosteric regulation.

2.6. Phenotypic/cellular Effects Caused by UCH-L3

The following assays are performed with cell lines, e.g. THP-1 (Tsuchiya, S. et al. (1980) Int. J. Cancer 26, 171-176), MonoMac 6 (Ziegler-Heitbrock, H. W. et al. (1988) Int. J. Cancer 41, 456-461) that are transiently or stably transfected with UCH-L3 and the read-outs are compared to mock-transfected cells. In addition, substances according to the invention that stimulate the activity of UCH-L3 are added.

### Production and Release of Cytokines

Monocytic/macrophage cell lines are stimulated with various stimuli, like 10 nM PMA, 20 ng/ml M-CSF, 20 ng/ml GM-CSF, 20 μg/ml LPS (from Salmonella minnesota Re595) at cell densities between 2.5 and 5×10⁵ cells/ml. Cells are harvested after 0, 1, 3, 6, 12, 24, 48, and 72 hours, and the supernatant frozen for further investigation; cells are washed with PBS, and resuspended in 400 μl of RLT buffer (from Qiagen RNeasy Total RNA Isolation Kit) with 143 mM β-mercaptoethanol, the DNA sheared with a 20 g needle for at least 5 times and stored at −70° C.

Stimulation of cells by cigarette smoke is performed by a smoke-enriched media. 100 ml RPMI media without supplements is perfused with the cigarette smoke of 2 cigarettes. The smoke of the cigarettes is pulled into a 50 ml syringe (about 20 volumes of a 50-ml volumes per cigarette) and then perfused into the media. Afterwards, the pH of the media is adjusted to 7.4, and the media is filter sterilized through a 0.2 μm filter. Cells are resuspended in smoke-enriched media and incubated for 10 minutes at 37° C. at a density of 1×10⁶ cells/ml. Then, cells are washed twice with RPMI 1640 and seeded in flasks or 24-well plates (MonoMac6) for the times indicated above.

Total RNAs are isolated with the Qiagen RNeasy Total RNA Isolation Kit (Qiagen) according to the manufacturer's protocol. Purified RNA is used for TaqMan analysis. The expression levels of cytokines, TNFα, IL-1β, IL-8, and IL-6, are measured.

### Detection of Secreted Cytokines

Proteins in the supernatants of the cultured and stimulated cells are precipitated by adding TCA to a final concentration of 10%. Precipitates are washed twice with 80% ethanol and pellets are resuspended in 50 mM Tris/HCl, pH 7.4, 10 mM MgCl₂, 1 mM EDTA. Protein concentration is determined via the Bradford method and 50 μg of each sample are loaded on 12% SDS polyacrylamide gels. Gels are blotted onto PVDF-membranes, blocked for 1 hour in 5% BSA in TBST, and incubated for 1 hour with commercially available antibodies against human TNFα, IL-1β, IL-8, and IL-6. After washing with TBST, blots are incubated with anti-human IgG conjugated to horseradish-peroxidase, washed again and developed with ECL chemiluminescence kit (Amersham). Intensity of the bands are visualized with BioMax X-ray films (Kodak) and quantified by densitometry.

### Detection of Secreted Matrix Metalloproteases and Other Proteases

The procedure is identical to the one used for cytokines. Antibodies used for Western blotting are against human MMP-1, MMP-7, MMP-9, and MMP-12.

### Activity of Secreted Matrix Metalloproteases

Protease activity is determined with a fluorescent substrate. Supernatants isolated from stimulated and unstimulated cells (described above) are incubated in a total volume of 50 μl with 1 μM of the substrate (Dabcyl-Gaba-Pro-Gln-Gly-Leu-Glu (EDANS)-Ala-Lys-NH2 (Novabiochem)) for 5 minutes at room temperature. Positive controls are performed with 125 ng purified MMP-12 per reaction. Protease activity is determined by fluorometry with an excitation at 320 nm and an emission at 405 nm.

In an alternative assay to determine proteolytic activity and cell migration, a chemotaxis (Boyden) chamber is used. In the wells of the upper part of the chamber cells (10⁵ cells per well) are plated on filters coated with an 8 μm layer of Matrigel (Becton Dickinson). In the lower compartment, chemoattractants like leukotriene B₄ (10 ng/ml), MCP-1 (10 ng/ml) are added to the media. After five days filters are removed, cells on the undersurface that have traversed the Matrigel are fixed with methanol, stained with the Diff-Quik staining kit (Dade Behring) and counted in three high power fields (400×) by light microscopy.

### Chemotaxis Assay

In order to determine chemotaxis, a 48 well chemotaxis (Boyden) chamber (Neuroprobe) is used. Cells are starved for 24 hours in RPMI media without FCS. Chemoattractants (50 ng/ml IL-8, 10 ng/ml MCP-1, 10 nM lipoxin A4) and substances according to the invention are diluted in RPMI media without FCS, and 30 μl is placed in the wells of the lower compartment. The upper compartment is separated from the lower compartment by a polycarbonate filter (pore size 8 μm). 50 μl of cell suspension (5×10⁴) are placed in the well of the upper compartment. The chamber is incubated for 5 hours at 37° C. in a humidified atmosphere with 5% CO₂. Then, the filter is removed; cells on the upper side are scraped off; cells on the downside are fixed for 5 minutes in methanol and stained with the Diff-Quik staining set (Dade Behring). Migrated cells are counted in three high-power fields (400×) by light microscopy.

### Adherence Assay

Cells are harvested, washed in PBS and resuspended (4×10⁶/ml) in PBS and 1 μM BCECF ((2′-7′-bis-(carboxyethyl)-5(6′)-carboxyfluorescein acetoxymethyl) ester, Calbiochem) and incubated for 20 minutes at 37° C. Cells are washed in PBS and resuspended (3.3×10⁶/ml) in PBS containing 0.1% BSA. 3×10⁵ cells (90 μl) are added to each well of a 96-well flat bottom plate coated with laminin (Becton Dickinson) and allowed to settle for 10 minutes. Substances according to the invention are added and plates are incubated for 20 minutes at 37° C. Cells are washed with PBS containing 0.1% BSA and adherent cells are solubilized with 100 μl of 0.025 M NaOH and 0.1% SDS. Quantification is performed by fluorescence measurement.

### Phagocytosis

Cell suspensions (2.5×10⁴ cells/ml) are seeded in 6-well plates with 5 ml of U937 or THP-1 or in 24-well plates with 2 ml of MonoMac6 and incubated for 1 hour at 37° C. in a humidified atmosphere with 5% CO₂ in the presence of substances according to the invention. 40 μl of a dispersed suspension of heat-inactivated Saccharomyces boulardii (20 yeast/cell) are added to each well. Cells are incubated for three more hours, washed twice with PBS and cytocentrifuged. The cytospin preparations are stained with May-Grünwald-Giemsa and phagocytosed particles are counted by light microscopy.

### Example 3

### HC3 subunit of the S20 proteasome complex

A gene that is identified as being up-regulated in COPD smokers compared to healthy smokers is the HC3 subunit of the S20 proteasome complex. This subunit recognizes proteins that are conjugated to ubiquitin and leads to their proteolytic degradation. Increased mRNA levels of the proteasome subunit HC3, SEQ ID NOs:7, 8 have been found in sepsis in human patients and in animal models (Tiao, G. et al. (1997) J. Clin. Invest. 99,163-168; Hobler, S. C. et al. (1999) Am. J. Physiol. 277, R434-R440).

Proteasome subunit HC3 is consistently found up-regulated (53%) in comparisons between COPD smokers and healthy smokers. This is shown by “fold change” values (Table 2 ). The p values for two separate groups comparing COPD smokers and healthy smokers are 0.17 and 0.02.

TABLE 2
Fold change values (FC) for comparisons between obstructed
smoker and healthy smokers. On average proteasome subunit
HC3 is up-regulated by 1.7 fold, the median is 2.1 fold

| comp | FC | comp | FC | comp | FC | comp | FC |
|---|---|---|---|---|---|---|---|
| 1 vs 2 | −1.2 | 5 vs 43 | 2.6 | 39 vs 57 | 4.2 | 68 vs 66 | 2.0 |
| 1 vs 37 | 2.6 | 5 vs 56 | 3.9 | 39 vs 58 | 1.8 | 68 vs 69 | 6.4 |
| 1 vs 43 | 2.4 | 5 vs 57 | 4.6 | 39 vs 62 | 3.0 | 68 vs 76 | 1.8 |
| 1 vs 56 | 3.4 | 5 vs 58 | 2.0 | 44 vs 2 | −3.3 | 68 vs 78 | 2.6 |
| 1 vs 57 | 4.2 | 5 vs 62 | 3.3 | 44 vs 37 | −1.2 | 70 vs 65 | −1.5 |
| 1 vs 58 | 1.8 | 6 vs 2 | −1.3 | 44 vs 43 | −1.2 | 70 vs 66 | −1.2 |
| 1 vs 62 | 3.0 | 6 vs 37 | 2.1 | 44 vs 56 | 1.1 | 70 vs 69 | 2.7 |
| 3 vs 2 | −1.1 | 6 vs 43 | 2.1 | 44 vs 57 | 1.4 | 70 vs 76 | −1.2 |
| 3 vs 37 | 2.6 | 6 vs 56 | 2.8 | 44 vs 58 | −1.7 | 70 vs 78 | 1.2 |
| 3 vs 43 | 2.5 | 6 vs 57 | 3.4 | 44 vs 62 | 1.0 | 71 vs 65 | 1.2 |
| 3 vs 56 | 3.5 | 6 vs 58 | 1.5 | 64 vs 65 | −1.2 | 71 vs 66 | 1.6 |
| 3 vs 57 | 4.1 | 6 vs 62 | 2.3 | 64 vs 66 | 1.1 | 71 vs 69 | 5.2 |
| 3 vs 58 | 1.8 | 39 vs 2 | −1.1 | 64 vs 69 | 3.3 | 71 vs 76 | 1.5 |
| 3 vs 62 | 2.8 | 39 vs 37 | 2.5 | 64 vs 76 | 1.0 | 71 vs 78 | 2.1 |
| 5 vs 2 | 1.0 | 39 vs 43 | 2.4 | 64 vs 78 | 1.5 | | |
| 5 vs 37 | 2.9 | 39 vs 56 | 3.3 | 68 vs 65 | 1.5 | | |

The protein is cloned and assays are performed in an analogous manner to the cloning and assays described hereinbefore.

What is claimed is:

1. A method for determining whether a substance is an activator or an inhibitor of a function of a UDD-protein comprising: (a) contacting the UDD-protein with a substance to be tested; and (b) measuring whether the function is inhibited or activated.

2. A method for determining whether a substance is an activator or an inhibitor of a function of a protein comprising: (a) contacting the protein with a substance to be tested, wherein the protein is a functionally equivalent variant, mutant or fragment of a UDD-protein; and (b) measuring whether the function is inhibited or activated.

3. The method according to claim 1 wherein the inhibition or activation of the function is measured directly.

4. The method according to claim 1 wherein the inhibition or activation of the function is measured indirectly.

5. The method according to claim 1 wherein the UDD-protein is a mammalian UDD-protein.

6. The method according to claim 5 wherein the UDD-protein is a human UDD-protein.

7. The method according to claim 1 wherein the method is performed using a cellular system.

8. The method according to claim 1 wherein the method is performed using a cell-free system.

9. The method according to claim 1 wherein the UDD-protein consists of an amino acid sequence selected from the group consisting of: SEQ ID NO:4 and SEQ ID NO:8.

10. The method according to claim 9 wherein the amino acid sequence is SEQ ID NO. 4.

11. The method according to claim 9 wherein the amino acid sequence is a functionally equivalent mutant, variant or fragment of SEQ ID NO. 4.

12. The method according to claim 9 wherein the amino acid sequence is SEQ ID NO. 8.

13. The method according to claim 9 wherein the amino acid sequence is a functionally equivalent mutant, variant or fragment of SEQ ID NO. 8.

14. The method according to claim 1 wherein the function is substrate binding.

15. A method for determining an expression level of a UDD-protein comprising: (a) determining the level of UDD-protein expressed in a hyperactivated macrophage; (b) determining the level of UDD-protein expressed in a non-hyperactivated macrophage; and (c) comparing the level of the UDD-protein expressed in step (a) to the level of the UDD-protein expressed in step (b), wherein a difference in the levels indicates a differentially expressed protein.

16. The method according to claim 15 wherein the hyperactivated macrophage is a mammalian macrophage and the non-hyperactivated macrophage is a mammalian macrophage.

17. The method according to claim 16 wherein the hyperactivated macrophage is a human macrophage and the non-hyperactivated macrophage is a human macrophage.

18. The method according to claim 15 wherein the UDD-protein consists of an amino acid sequence selected from the group consisting of: SEQ ID NO:4 and SEQ ID NO:8.

19. The method according to claim 18 wherein the amino acid sequence is SEQ ID NO:4.

20. The method according to claim 18 wherein the amino acid sequence is a functionally equivalent variant, mutant or fragment of SEQ ID NO:4.

21. The method according to claim 18 wherein the amino acid sequence is SEQ ID NO:8.

22. The method according to claim 18 wherein the amino acid sequence is a functionally equivalent variant, mutant or fragment of SEQ ID NO:8.

23. A for diagnosing or monitoring a chronic inflammatory airway disease comprising: (a) determining the level of UDD-protein expressed in a hyperactivated macrophage; (b) determining the level of UDD-protein expressed in a non-hyperactivated macrophage; and (c) comparing the level of the UDD-protein expressed in step (a) to the level of the UDD-protein expressed in step (b), wherein a difference in the levels indicates a differentially expressed protein.

24. The method according to claim 23 wherein the chronic inflammatory airway disease is selected from the group consisting of: chronic bronchitis and COPD.

25. The method according to claim 15 wherein the method is performed using a macrophage or a part thereof obtainable from a site of inflammation.

26. A substance determined to be an activator or an inhibitor of a UDD-protein.

27. A substance determined to be an activator or an inhibitor of a UDD-protein according to the method of claim 1.

28. A substance for the treatment for a disease wherein the substance is an activator or an inhibitor of a UDD-protein.

29. The substance according to claim 28 wherein the disease is a chronic inflammatory airway disease.

30. The substance according to claim 29 wherein the chronic inflammatory airway disease is selected from the group consisting of: chronic bronchitis and COPD.

31. A pharmaceutical composition comprising at least one substance which is an activator or an inhibitor of a UDD-protein; and a pharmaceutical carrier.

32. A pharmaceutical composition comprising at least one substance which is an activator or an inhibitor of a UDD-protein according to the method of claim 1; and a pharmaceutical carrier.

33. A method for treating a chronic inflammatory airway disease comprising:
administering to a being in need of such treatment an effective amount of a pharmaceutical composition comprising at least one substance determined to be an activator or an inhibitor of a UDD-protein.

34. The method according to claim 33 for treating a mammal.

35. The method according to claim 33 for treating a human.

36. The method according to claim 33 for treating a chronic inflammatory airway disease selected from the group consisting of: chronic bronchitis and COPD.

37. A method for treating a chronic inflammatory airway disease comprising:
administering to a being in need of such treatment an effective amount of a pharmaceutical composition comprising at least one substance determined to be an activator or an inhibitor of a UDD-protein according to the method of claim 1.

38. A method for selectively modulating a UDD-protein in a macrophage, comprising administering a substance determined to be an activator or an inhibitor of a UDD-protein.

39. A method according to claim 38 wherein the macrophage is involved in a chronic inflammatory airway disease

40. The method according to claim 39 wherein the chronic inflammatory airway disease is selected from the group consisting of: chronic bronchitis and COPD.

41. A method for selectively modulating a UDD-protein in a macrophage, comprising administering a substance determined to be an activator or an inhibitor of a UDD-protein according to the method of claim 1.
